SEQUENCE LISTING (19 sequences)

SEQ ID NO: 1 - protein, 383 aa, Unknown
  Human Cervical Cancer Cell Lines (HELA)
MGLGPVFLLLAGIFPFAPPGAAAEPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWQTFHVSAVAAAAIFVIIIFYVRCCKKKTSAAEGPELVSLQVLDQHPVGTSDHRDATQLGFQPLMSDLGSTGSTEGA

SEQ ID NO: 2 - protein, 383 aa, Unknown
  Human Cervical Cancer Cell Lines (HELA)
MGLGRVLLFLAVAFPFAPPAAAAEPHSLRYNLMVLSQDGSVQSGFLAEGHLDGQPFLRYDRQKRRAKPQGQWAENVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKALVLQSQRTDFPYVSAAMPCFVIIIILCVPCCKKKTSAAEGPELVSLQVLDQHPVGTGDHRDAAQLGFQPLMSATGSTGSTEGT

SEQ ID NO: 3 - protein, 244 aa, Unknown
  Human Cervical Cancer Cell Lines (HELA)
MAAAASPAFLLCLPLLHLLSGWSRAGWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPGTTQPKAMATTLSPWSLLIIFLCFILAGR

SEQ ID NO: 4 - protein, 246 aa, Unknown
  Human Cervical Cancer Cell Lines (HELA)
MAAAAATKILLCLPLLLLLSGWSRAGRADPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPLAMSSGTTQLRATATTLILCCLLIILPCFILPGI

SEQ ID NO: 5 - protein, 244 aa, Unknown
  Human Cervical Cancer Cell Lines (HELA)
MAAAASPAILPRLAILPYLLFDWSGTGRADAHSLWYNFTIIHLPRHGQQWCEVQSQVDQKNFLSYDCGSDKVLSMGHLEEQLYATDAWGKQLEMLREVGQRLRLELADTELEDFTPSGPLTLQVRMSCECEADGYIRGSWQFSFDGRKFLLFDSNNRKWTVVHAGARRMKEKWEKDSGLTTFFKMVSMRDCKSWLRDFLMHRKKRLEPTAPPTMAPGLAQPKAIATTLSPWSFLIILCFILPGI

SEQ ID NO: 6 - protein, 263 aa, Unknown
  Human Cervical Cancer Cell Lines (HELA)
MRRISLTSSPVRLLLFLLLLLIALEIMVGGHSLCFNFTIKSLSRPGQPWCEAQVFLNKNLFLQYNSDNNMVKPLGLLGKKVYATSTWGELTQTLGEVGRDLRMLLCDIKPQIKTSDPSTLQVEMFCQREAERCTGASWQFATNGEKSLLFDAMNMTWTVINHEASKIKETWKKDRGLEKYFRKLSKGDCDHWLREFLGHWEAMPEPTVSPVNASDIHWSSSSLPDRWIILGAFILLVLMGIVLICVWWQNGEWQAGLWPLRTS

SEQ ID NO: 7 - DNA, 1152 bp, Unknown
  Human Cervical Cancer Cell Lines (HELA)
atggggctgggcccggtcttcctgcttctggctggcatcttcccttttgcacctccgggagctgctgctgagccccacagtcttcgttataacctcacggtgctgtcctgggatggatctgtgcagtcagggtttctcactgaggtacatctggatggtcagcccttcctgcgctgtgacaggcagaaatgcagggcaaagccccagggacagtgggcagaagatgtcctgggaaataagacatgggacagagagaccagagacttgacagggaacggaaaggacctcaggatgaccctggctcatatcaaggaccagaaagaaggcttgcattccctccaggagattagggtctgtgagatccatgaagacaacagcaccaggagctcccagcatttctactacgatggggagctcttcctctcccaaaacctggagactaaggaatggacaatgccccagtcctccagagctcagaccttggccatgaacgtcaggaatttcttgaaggaagatgccatgaagaccaagacacactatcacgctatgcatgcagactgcctgcaggaactacggcgatatctaaaatccggcgtagtcctgaggagaacagtgccccccatggtgaatgtcacccgcagcgaggcctcagagggcaacattaccgtgacatgcagggcttctggcttctatccctggaatatcacactgagctggcgtcaggatggggtatctttgagccacgacacccagcagtggggggatgtcctgcctgatgggaatggaacctaccagacctgggtggccaccaggatttgccaaggagaggagcagaggttcacctgctacatggaacacagcgggaatcacagcactcaccctgtgccctctgggaaagtgctggtgcttcagagtcattggcagacattccatgtttctgctgttgctgctgctgctatttttgttattattattttctatgtccgttgttgtaagaagaaaacatcagctgcagagggtccagagctcgtgagcctgcaggtcctggatcaacacccagttgggacgagtgaccacagggatgccacacagctcggatttcagcctctgatgtcagatcttgggtccactggctccactgagggcgcctag

SEQ ID NO: 8 - DNA, 1152 bp, Unknown
  Human Cervical Cancer Cell Lines (HELA)
atggggctgggccgggtcctgctgtttctggccgtcgccttcccttttgcacccccggcagccgccgctgagccccacagtcttcgttacaacctcatggtgctgtcccaggatggatctgtgcagtcagggtttctcgctgagggacatctggatggtcagcccttcctgcgctatgacaggcagaaacgcagggcaaagccccagggacagtgggcagaaaatgtcctgggagctaagacctgggacacagagaccgaggacttgacagagaatgggcaagacctcaggaggaccctgactcatatcaaggaccagaaaggaggcttgcattccctccaggagattagggtctgtgagatccatgaagacagcagcaccaggggctcccggcatttctactacgatggggagctcttcctctcccaaaacctggagactcaagaatcgacagtgccccagtcctccagagctcagaccttggctatgaacgtcacaaatttctggaaggaagatgccatgaagaccaagacacactatcgcgctatgcaggcagactgcctgcagaaactacagcgatatctgaaatccggggtggccatcaggagaacagtgccccccatggtgaatgtcacctgcagcgaggtctcagagggcaacatcaccgtgacatgcagggcttccagcttctatccccggaatatcacactgacctggcgtcaggatggggtatctttgagccacaacacccagcagtggggggatgtcctgcctgatgggaatggaacctaccagacctgggtggccaccaggattcgccaaggagaggagcagaggttcacctgctacatggaacacagcgggaatcacggcactcaccctgtgccctctgggaaggcgctggtgcttcagagtcaacggacagactttccatatgtttctgctgctatgccatgttttgttattattattattctctgtgtcccttgttgcaagaagaaaacatcagcggcagagggtccagagcttgtgagcctgcaggtcctggatcaacacccagttgggacaggagaccacagggatgcagcacagctgggatttcagcctctgatgtcagctactgggtccactggttccactgagggcacctag

SEQ ID NO: 9 - DNA, 735 bp, Unknown
  Human Cervical Cancer Cell Lines (HELA)
atggcagcggccgccagccccgcgttccttctgtgcctcccgcttctgcacctgctgtctggctggtcccgggcaggatgggtcgacacacactgtctttgctatgacttcatcatcactcctaagtccagacctgaaccacagtggtgtgaagttcaaggcctggtggatgaaaggccttttcttcactatgactgtgttaaccacaaggccaaagcctttgcttctctggggaagaaagtcaatgtcacaaaaacctgggaagaacaaactgaaacactaagagacgtggtggatttccttaaagggcaactgcttgacattcaagtggagaatttaatacccattgagcccctcaccctgcaggccaggatgtcttgtgagcatgaagcccatggacacggcagaggatcttggcagttcctcttcaatggacagaagttcctcctctttgactcaaacaacagaaagtggacagcacttcatcctggagccaagaagatgacagagaagtgggagaagaacagggatgtgaccatgttcttccagaagatttcactgggggattgtaagatgtggcttgaagaatttttgatgtactgggaacaaatgctggatccaacaaaaccaccctctctggccccaggcacaacccaacccaaggccatggccaccaccctcagtccctggagccttctcatcatcttcctctgcttcattctagctggcagatga

SEQ ID NO: 10 - DNA, 741 bp, Unknown
  Human Cervical Cancer Cell Lines (HELA)
atggcagcagccgccgctaccaagatccttctgtgcctcccgcttctgctcctgctgtccggctggtcccgggctgggcgagccgaccctcactctctttgctatgacatcaccgtcatccctaagttcagacctggaccacggtggtgtgcggttcaaggccaggtggatgaaaagacttttcttcactatgactgtggcaacaagacagtcacacctgtcagtcccctggggaagaaactaaatgtcacaacggcctggaaagcacagaacccagtactgagagaggtggtggacatacttacagagcaactgcgtgacattcagctggagaattacacacccaaggaacccctcaccctgcaggccaggatgtcttgtgagcagaaagctgaaggacacagcagtggatcttggcagttcagtttcgatgggcagatcttcctcctctttgactcagagaagagaatgtggacaacggttcatcctggagccagaaagatgaaagaaaagtgggagaatgacaaggttgtggccatgtccttccattacttctcaatgggagactgtataggatggcttgaggacttcttgatgggcatggacagcaccctggagccaagtgcaggagcaccactcgccatgtcctcaggcacaacccaactcagggccacagccaccaccctcatcctttgctgcctcctcatcatcctcccctgcttcatcctccctggcatctga

SEQ ID NO: 11 - DNA, 735 bp, Unknown
  Human Cervical Cancer Cell Lines (HELA)
atggcagcggccgccagccccgcgatccttccgcgcctcgcgattcttccgtacctgctattcgactggtccgggacggggcgggccgacgctcactctctctggtataacttcaccatcattcatttgcccagacatgggcaacagtggtgtgaggtccagagccaggtggatcagaagaattttctctcctatgactgtggcagtgacaaggtcttatctatgggtcacctagaagagcagctgtatgccacagatgcctggggaaaacaactggaaatgctgagagaggtggggcagaggctcagactggaactggctgacactgagctggaggatttcacacccagtggacccctcacgctgcaggtcaggatgtcttgtgagtgtgaagccgatggatacatccgtggatcttggcagttcagcttcgatggacggaagttcctcctctttgactcaaacaacagaaagtggacagtggttcacgctggagccaggcggatgaaagagaagtgggagaaggatagcggactgaccaccttcttcaagatggtctcaatgagagactgcaagagctggcttagggacttcctgatgcacaggaagaagaggctggaacccacagcaccacccaccatggccccaggcttagctcaacccaaagccatagccaccaccctcagtccctggagcttcctcatcatcctctgcttcatcctccctggcatctga

SEQ ID NO: 12 - DNA, 792 bp, Unknown
  Human Cervical Cancer Cell Lines (HELA)
atgcgaagaatatccctgacttctagccctgtgcgccttcttttgtttctgctgttgctactaatagccttggagatcatggttggtggtcactctctttgcttcaacttcactataaaatcattgtccagacctggacagccctggtgtgaagcgcaggtcttcttgaataaaaatcttttccttcagtacaacagtgacaacaacatggtcaaacctctgggcctcctggggaagaaggtatatgccaccagcacttggggagaattgacccaaacgctgggagaagtggggcgagacctcaggatgctcctttgtgacatcaaaccccagataaagaccagtgatccttccactctgcaagtcgagatgttttgtcaacgtgaagcagaacggtgcactggtgcatcctggcagttcgccaccaatggagagaaatccctcctctttgacgcaatgaacatgacctggacagtaattaatcatgaagccagtaagatcaaggagacatggaagaaagacagagggctggaaaagtatttcaggaagctctcaaagggagactgcgatcactggctcagggaattcttagggcactgggaggcaatgccagaaccgacagtgtcaccagtaaatgcttcagatatccactggtcttcttctagtctaccagatagatggatcatcctgggggcattcatcctgttagttttaatgggaattgttctcatctgtgtctggtggcaaaatggtgagtggcaggctggtctctggcccttgaggacgtcttag

SEQ ID NO: 13 - protein, 138 aa, Artificial sequence
  Synthesized
MEFGLSWLFLVAILKGVQCEVQLVESGGGLVQPGGSLRLSCAVSGFSLTNYGVHWVRQATGKGLEWLGVIWSGGNTDYNTPFTSRLTISKENAKNSLYLQMNSLRAGDTAVYYCARALTYYDYEFAYWGQGTMVTVSS

SEQ ID NO: 14 - protein, 94 aa, Artificial sequence
  Synthesized
PSVFPLAPSSKSTSGGTAALGCLVKDYFPEPVTVSWNSGALTSGVHTFPAVLQSSGLYSLSSVVTVPSSSLGTQTYICNVNHKPSNTKVDKKVE

SEQ ID NO: 15 - protein, 127 aa, Artificial sequence
  Synthesized
MEAPAQLLFLLLLWLPDTTGEIVLTQSPATLSLSPGERATLSCRASQSIGTNIHWYQQRPGQAPRLLIKYASESISGIPARFSGSGSGTDFTLTISSLEPEDFAVYYCQQNNNWPTTFGGGTKVEIK

SEQ ID NO: 16 - protein, 100 aa, Artificial sequence
  Synthesized
APSVFIFPPSDEQLKSGTASVVCLLNNFYPREAKVQWKVDNALQSGNSQESVTEQDSKDSTYSLSSTLTLSKADYEKHKVVACEVTHQGLSSPVTKSFNR

SEQ ID NO: 17 - DNA, 87 bp, Artificial sequence
  Synthesized
aagcttgggggcggcggaagtggaggaggggggtctggcggaggcggcagtggcggaggcggaagtggaggaggagggtcaactagt

SEQ ID NO: 18 - DNA, 745 bp, Artificial sequence
  Synthesized
ggggtaccccgcgcctaggcgcatggagttcggacttagctggctcttcctggtggcaatcctgaagggggtgcaatgcgaggtgcagcttgtcgagtctggaggcggcctggtccagcctggagggtctttgagattgtcctgcgctgtgtcagggttctccctgactaactacggagtccactgggtcaggcaggctacagggaaagggctggaatggctgggcgtgatttggagtggaggcaatactgattacaacacaccctttaccagccgcctgaccatctccaaggagaacgccaagaacagcctgtacctccagatgaactccctcagggccggcgacaccgccgtgtactactgtgcccgggccctcacatactacgactacgagtttgcctattggggccagggcacaatggtgaccgtgagcagcgcgccagaatggcgcccctcagtgttccctctcgctccaagttccaagtccaccagcggaggcacagcagcccttgggtgtttggtgaaagattacttccccgagccagtgaccgtgagttggaactccggcgccctgactagcggggtccatacatttcccgccgtcctgcaatcaagcgggctgtactccctcagcagcgtcgtgactgtgcctagctcttctctgggcacacagacatatatctgcaatgtcaaccacaagccctccaacaccaaggtggacaagaaggtggaaccgctcgagcgg

SEQ ID NO: 19 - DNA, 747 bp, Artificial sequence
  Synthesized
ggggtaccccgcgatatccgatggaggcaccagctcaactcttgtttctcctcctgctctggttgcctgacaccacaggcgagatcgtgctgacccagtcccctgctacactgtccctgtctccaggagaaagagccactctgtcttgcagggcctcacagagtatcgggactaacatccactggtatcagcagcgccctgggcaggcccccaggcttcttattaagtacgccagcgagagcatcagcggaattcccgcccggttctccggcagcgggagcggcactgactttacactgacaatttcctccctggagcccgaggatttcgccgtgtactactgtcagcagaacaacaattggcccacaaccttcggtggaggcaccaaagtcgagattaagccgctcgagcggcccaagcttggggctccttcagtgttcatctttcctccctccgacgaacagctcaagtccggcacagcaagtgtggtgtgtttgctgaacaacttctacccaagggaggccaaggtgcagtggaaggtggacaacgcccttcagtctggaaatagccaggagtctgtgaccgagcaggatagtaaggactccacctacagcctgagcagcacactgacactgagcaaggccgattatgagaaacacaaagtcgtcgcctgcgaggtcactcatcaagggctctccagccccgtcactaagtcctttaaccggccttaattaaggggactagtcc

(19) United States
(12) Patent Application Publication
(10) Pub. No.: US 2018/0201689 A1
(43) Pub. Date: Jul. 19, 2018

(54) Antibody Fusion Protein and Preparation Method and Use Thereof

(71) Applicant: Institute of Microbiology, Chinese Academy of Sciences, Beijing (CN)

(72) Inventors: Min Fang, Beijing (CN); Wei Jiang, Beijing (CN); Xiuling Gu, Beijing (CN); Dongfang Wang, Beijing (CN); Kaili Li, Beijing (CN)

(21) Appl. No.: 15/745,031

(22) PCT Filed: Jun. 30, 2016
(86) PCT No.: PCT/CN2016/087967

§ 371 Date: Jan. 15, 2018

(30) Foreign Application Priority Data
Aug. 18, 2015 (CN) 201510509687.6

(51) Int. Cl. C07K 16/30; A61K 39/395; C12N 15/63

(52) U.S. Cl. CPC C07K 16/30; A61K 39/39558; C12N 15/63; C12N 2330/51; A61K 2039/605; C07K 2317/55; C07K 2317/92; C07K 2319/035; C07K 2317/32

(57) ABSTRACT

The present invention provides an antibody fusion protein, comprising an antitumor antigen-specific antibody or a Fab fragment thereof, a single domain antibody or single chain antibody, and further comprising a human NKG2D ligand or ligand fragment. The human NKG2D ligand or ligand fragment and the antitumor antigen-specific antibody or Fab fragment thereof and the single domain antibody or single chain antibody are mutually connected by a linker peptide.

### TECHNICAL FIELD

The present invention relates to field of immune technology, specifically relates to an antibody fusion protein and preparation method and use thereof.

### BACKGROUND

Antibody drugs are drugs prepared by antibody engineering technology based on cell engineering technology and genetic engineering technology. The drugs have the advantages of high specificity, homogeneous properties, and directional preparation for specific targets. Now antibody drugs have become one of the most successful and important strategies for the treatment of hematologic malignancies and solid tumors.

The anti-tumor antibody drugs can be divided into 4 categories according to the structure: 1. antibody or antibody fragment; 2. bispecific antibody or trifunctional antibody; 3. antibody conjugate; 4. antibody fusion protein.

Wherein, the antibody fusion protein is prepared by fussing gene of antibodies (or antibody fragments) to that of the other effector proteins,

constructing fusion expression vector, and then producing fusion proteins in an appropriate expression system.

At present, T cells activated as immune effector cell are commonly used in tumor-targeted therapy for the following reasons:

(1) T cell population has the immunological memory cell; (2) tumor specific infiltrating T lymphocytes exist in tumor tissue; (3) studies in vivo and in vitro have shown that killer cell activated by anti CD3 antibody (CD3AK) has a stronger cytotoxic effect than that activated by IL-2 (LAK). Targeting TCR/CD3 complex or CD2 bispecific antibody has the targeting ability for all the T cells, which is not limited by the MHC. However, costimulatory signals are required for full T cell activation, CD28/B7 interaction plays an important role in enhancing the production of IL-2 and up-regulating high-affinity IL-2 receptor. In addition to CD28, CD2, LFA-1, CD5, ICAM-1, CD40 and cytokines such as IL-2, tumor necrosis factor (TNF) and so forth also affect the activation of T cells. So, the costimulatory signals required in T cell activation, drug combination realized through cytokine and costimulatory signal pathway, and a minimum degree of side effects when combining drugs should all be fully taken into consideration in clinical medication.

There are numerous reports that NK cells are referred to as immune effector cell used in a tumor targeted therapy. NK cells are natural and Non-MHC Restricted cytotoxic lymphocytes, of which the activity is determined by signal balance mediated by a series of inhibitory and activating receptors which are expressed on the surface of the NK cells. Now the molecules used in the activation of NK cell are often FcγRIII (CD16). However, in addition to the surface of NK cell, CD16 is also expressed in monocytes/macrophages and dendritic cells, then a wide activation of these cells will have strong side effects.

### SUMMARY

The present invention provides an antibody fusion protein and preparation method and use thereof. Not only can the antibody fusion protein achieve high efficiency and specificity in killing tumor cells, but also it can avoid toxic side effects and reduce the heterology of antibody.

The present invention provides an antibody fusion protein, comprising an antitumor antigen-specific antibody or a Fab fragment thereof, a single domain antibody or single chain antibody, and further comprising a human NKG2D ligand or ligand fragment;

Said human NKG2D ligand or ligand fragment and the antitumor antigen-specific antibody or Fab fragment thereof and the single domain antibody or single chain antibody are mutually connected by a linker peptide.

The above said antibody fusion protein, wherein, the amino acid sequence of said human NKG2D ligand are shown in from SEQ ID NO: 1 to SEQ ID NO: 6.

The above said antibody fusion protein, wherein, the coding gene sequence of said human NKG2D ligand are shown in from SEQ ID NO: 7 to SEQ ID NO: 12.

The above said antibody fusion protein, wherein, said antitumor antigen-specific antibody is a specific antibody targeting human tumor.

In another aspect, the invention also provides a recombinant vector, a recombinant cell, a recombinant bacterium, or an expression cassette, which contains the coding gene of any of said antibody fusion protein.

In another aspect, the invention also provides the preparation method of any of said antibody fusion protein, comprising:

Insert the heavy chain coding gene, NKG2D ligand coding gene and linker peptide coding gene of said antibody fusion protein into pBluescript II SK (+) vector to construct the pBS-SK-H plasmid;

Insert the light chain coding gene of said antibody fusion protein into pBluescript II SK (+) vector to construct the pBS-SK-L plasmid;

(c) Cleave the pBS-SK-H plasmid by enzyme to obtain vector fragment, which contains the heavy chain coding gene, NKG2D ligand coding gene and linker peptide coding gene of said antibody fusion protein;

(d) Cleave the pBS-SK-L plasmid by enzyme to obtain vector fragment, which contains the light chain coding gene of said antibody fusion protein;

(e) Insert the vector fragments obtained in the step (c) and the step (d) into expression vector to obtain the recombinant expression vector;

(f) Transform the recombinant expression vector obtained in the step (e) into recipient cell to express said antibody fusion protein.

The above said preparation method, wherein, the expression vector in said step (e) is pcDNA3.0-FLAG.

The above said preparation method, wherein, the recipient cell in said step (f) is 293T cell.

Method for preparing the above antibody fusion protein is just for example, the expression vector and the recipient cell can be properly selected, and any expression vector or recipient cell that can achieve the purpose of this invention can be used.

In another aspect, the invention also provides a use of any of the above antibody fusion protein in preparation of anti-tumor drugs.

In another aspect, the invention also provides drugs containing any of the above antibody fusion protein.

The advantages of the invention are as follows:

In this invention, the corresponding ligand (NKG2DL) of NK cell activating receptor NKG2D and the antibody of targeting tumor associated antigen are combined to form the fusion protein, wherein, the tumor associated antigen is used for targeting, NK cell is activated by NKG2DL, and NK cell activating receptor NKG2D can be expressed on the surface of all the NK cell. Since the expression of NKG2D has a strong specificity, NK cell can specifically kill tumor cells by combining NKG2DL with NK cell activating receptor NKG2D to activate the NK cell and thus promote the cytotoxicity of NK cell.

The invention uses the natural ligand of NKG2D rather than the antibody thereof to activate the related signal pathways, which avoid the toxic side effects when FcγR expressed cells are widely activated, thus efficiently and specifically activates the function of NK cell killing tumor cells.

The fusion protein of the present invention uses ligand of human source as NKG2D ligand, and avoids the problem of human antimouse antibody (HAMA) when using mouse source protein, thus reduces the heterology of the antibody.

### BRIEF DESCRIPTION OF THE DRAWINGS

FIG. 1 shows the structure diagram of antibody fusion protein in the embodiment of the invention.

FIG. 2 shows the structure diagram of pBluescript II SK (+) vector used in the embodiment of the invention.

FIG. 3 shows the structure diagram of heavy chain and light chain of antibody fusion protein in the embodiment of the invention.

FIG. 4 shows the structure diagram of expression vector pcDNA3.1-FLAG used in the embodiment of the invention.

FIG. 5 shows the identification figure of the successfully constructing pBS-SK-L of light chain in the embodiment of the invention.

FIG. 6 shows the identification figure of the successfully constructing pBS-SK-Linker of heavy chain in the embodiment of the invention.

FIG. 7 shows the identification figure of the successfully constructing pBS-SK-VH-CH1-linker of heavy chain in the embodiment of the invention.

FIG. 8 shows the identification figure of the successfully constructing pBS-SK-H of heavy chain in the embodiment of the invention (Take MICA as an example).

FIG. 9 shows the identification figure of the successfully constructing pcDNA3.1-FLAG-L of light chain in the embodiment of the invention.

FIG. 10 shows the identification figure of the successfully constructing pcDNA3.1-FLAG-H of heavy chain in the embodiment of the invention (Take MICA as an example).

FIG. 11 shows the expression identification figure of the pcDNA3.1-FLAG-L protein of light chain in the embodiment of the invention.

FIG. 12 shows the expression identification figure of the pcDNA3.1-FLAG-H protein of heavy chain in the embodiment of the invention (Take MICA as an example).

FIG. 13 shows the expression identification figure of EGFR on the surface of Hep3B cell strains in the embodiment of the invention.

FIG. 14 shows the expression identification figure of MICA on the surface of Hep3B cell strains in the embodiment of the invention.

FIG. 15 shows the killing efficiency of NK cells on tumor cell Hep3B when antibody fusion protein exists (Take anti EGFR antibody-MICA fusion protein as an example).

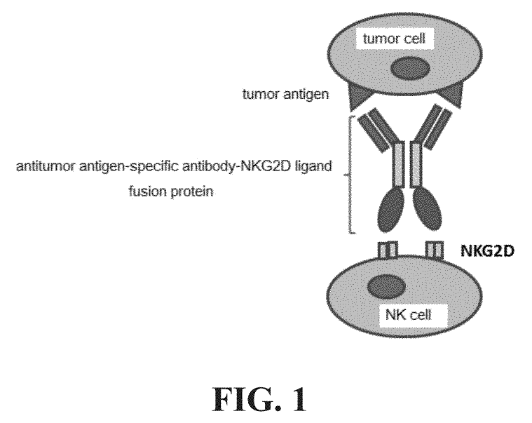

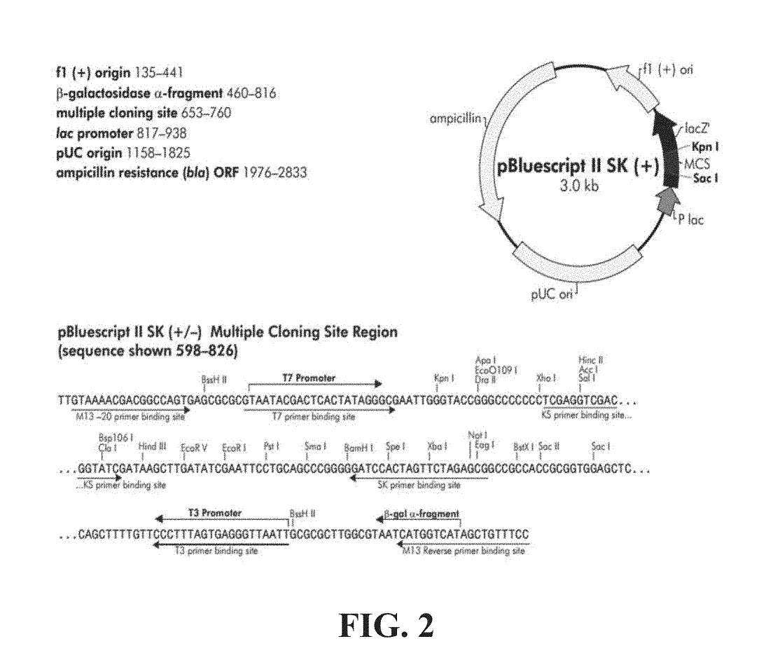

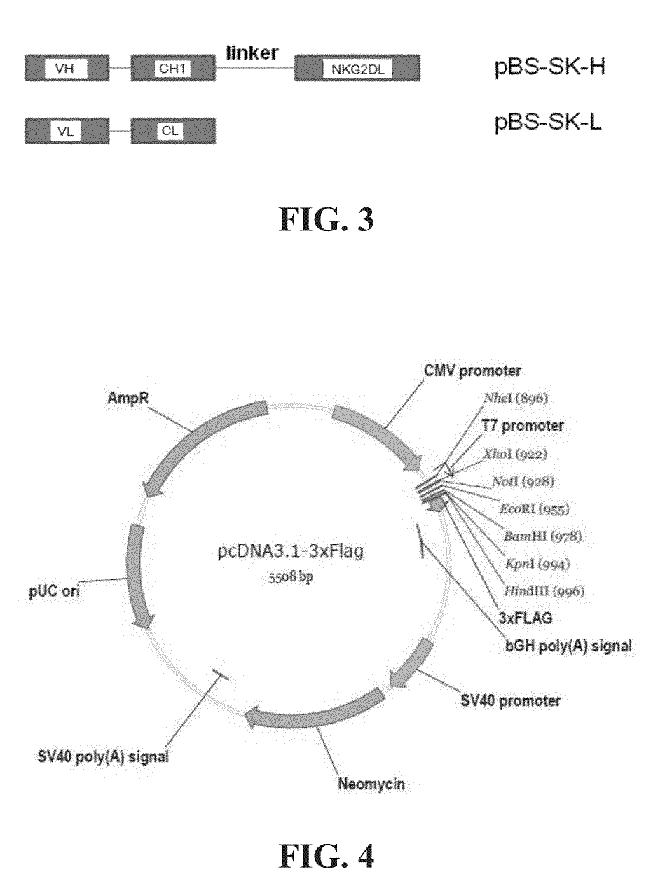

### DETAILED DESCRIPTION

In order to describe the present invention in more detail, the following examples and figures are provided in order to better understand the solution of the present invention and its advantages of various aspects.

The experiment methods used in the following examples are all conventional methods unless special version.

The materials and reagents used in the following examples are all commercial available unless special version.

### Example 1, Construction of pBS-SK-L Plasmid

Nucleotide sequence KpnI-EcoRV-VL-XhoI-HINDIII-CL-PacI-SpeI (nucleotide sequence is shown as SEQ ID NO:19) is synthesized and 747 bp in full length, in which KpnI cleavage site, EcoRV cleavage site, variable region sequence VL of antibody light chain (amino acid sequence is shown as SEQ ID NO:15), XhoI cleavage site, HindIII cleavage site, constant region CL of antibody light chain (amino acid sequence is shown as SEQ ID NO:16), Pad cleavage site and SpeI cleavage site are included.

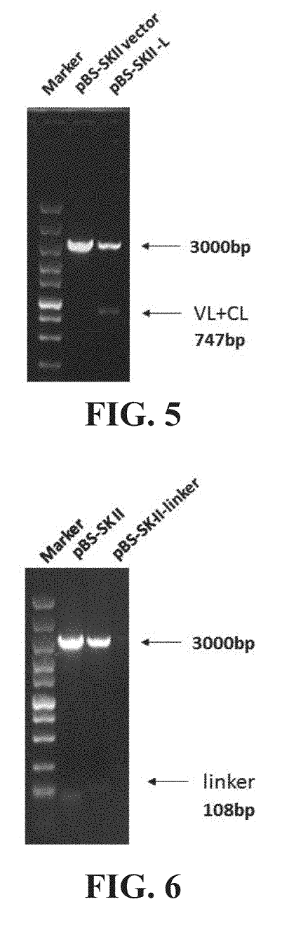

Inserting the above nucleotide sequence which has been cleaved by KpnI and SpeI into the vector pBluescript II SK (+) which has been cleaved by the same enzyme, pBS-SK-L is constructed and then verified by PCR (FIG. 5).

### Example 2, Construction of pBS-SK-H Plasmid

a. The nucleotide sequence fragment of linker peptide is inserted into the cleavage site of the vector pBluescript II SK (+) (purchased from Stratagen Co.) between HindIII and KpnI (nucleotide sequence of linker is shown as SEQ ID NO: 17), the vector pBS-SK-Linker is constructed and then verified by PCR (FIG. 6).

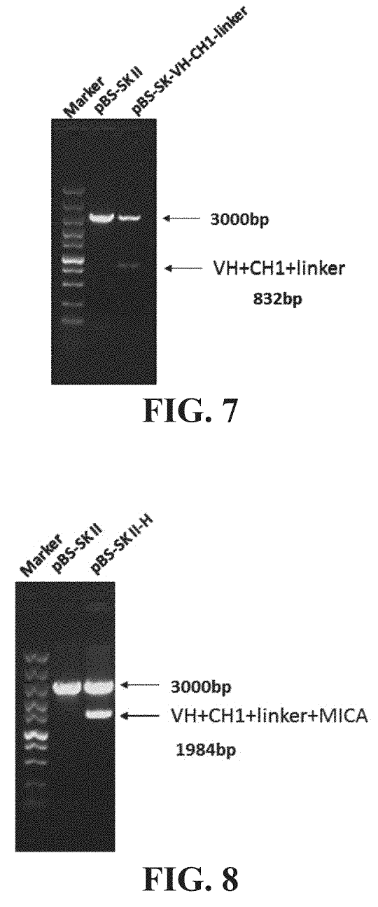

b. Nucleotide sequence KpnI-AvrII-VH-BglI-CH1-XhoI (nucleotide sequence is shown as SEQ ID NO: 18) is synthesized and 745 bp in full length, in which KpnI cleavage site, AvrII cleavage site, variable region sequence VH of antibody heavy chain (amino acid sequence is shown as SEQ ID NO: 13), BglI cleavage site, constant region CH1 of antibody heavy chain (amino acid sequence is shown as SEQ ID NO: 14) and XhoI cleavage site are included. Inserting the above nucleotide sequence which has been cleaved by KpnI and XhoI into the vector pBS-SK-Linker which has been cleaved by the same enzyme, pBS-SK-VH-CH1-linker is constructed and then verified by PCR (FIG. 7).

c. Six of the ligand coding gene sequence of NKG2D (the six ligand coding gene sequence of NKG2D are shown as from SEQ ID NO:7 to SEQ ID NO:12) are respectively cloned into the vector BS-SK-VH-CH1-linker, between two cleavage sites of XbaI and SacII, six different kinds of pBS-SK-H are constructed and then verified by PCR (FIG. 8).

### Example 3, Construction of the Expression Vector of Antibody Fusion Protein

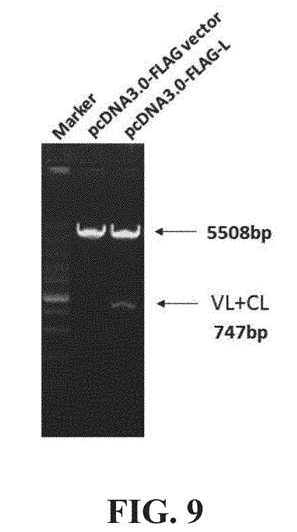

a. KpnI-EcoRV-VL-XhoI-HINDIII-CL-PacI-SpeI (nucleotide sequence is shown as SEQ ID NO:19), which is obtained from pBS-SK-L plasmid in Example 1, is cloned into the vector pcDNA3.1-FLAG (purchased from Thermofisher Co.) by PCR cloning method. Wherein, the cleavage sites are KpnI and NotI, the length of the sequence is 747 bp. Light chain expression plasmid pcDNA3.1-FLAG-L is constructed and then verified by PCR (FIG. 9).

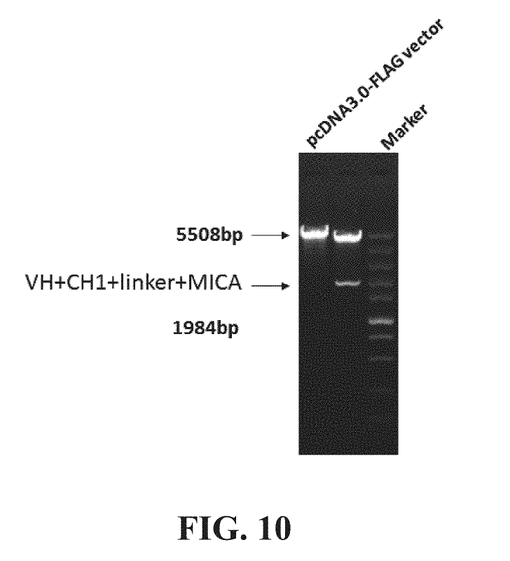

b. Six different kinds of VH-CH1-linker-NKG2DL, which are obtained from the six pBS-SK-H plasmids in Example 2, are respectively cloned into the vector pcDNA3.1-FLAG (purchased from Thermofisher Co.) by PCR cloning method. Wherein, the cleavage sites are NheI and NotI. Heavy chain expression plasmid pcDNA3.1-FLAG-H are constructed and then verified by PCR (FIG. 10).

### Example 4, Antibody Fusion Protein Expressing in 293T Cell

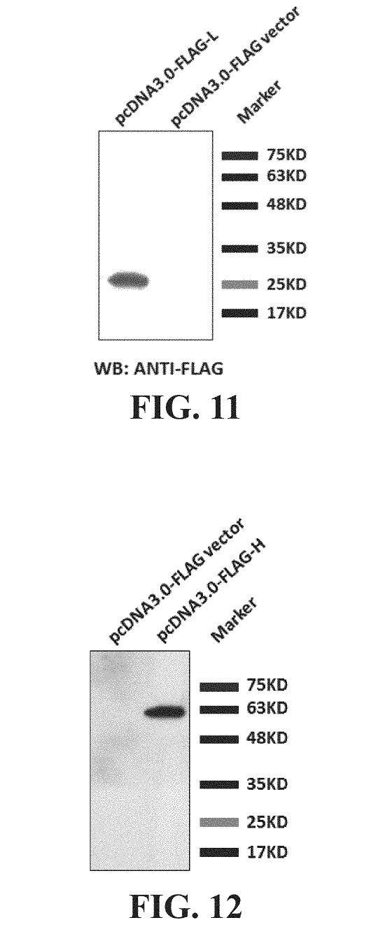

a. Light chain expression plasmid pcDNA3.1-FLAG-L is transfected into 293T cell by lipofectamine 2000 (purchased from US ATCC cell bank). The total cellular protein is collected after transfecting for 24 hours, and the expression of the target protein is detected by immunoblotting method (FIG. 11);

b. Heavy chain expression plasmid pcDNA3.1-FLAG-H is transfected into 293T cell by lipofectamine 2000 (purchased from US ATCC cell bank). The total cellular protein is collected after transfecting for 24 hours, and the expression of the target protein is detected by immunoblotting method (FIG. 12);

### Example 5, Activity Detection of Antibody Fusion Protein

In vitro identification of activity of fusion protein:

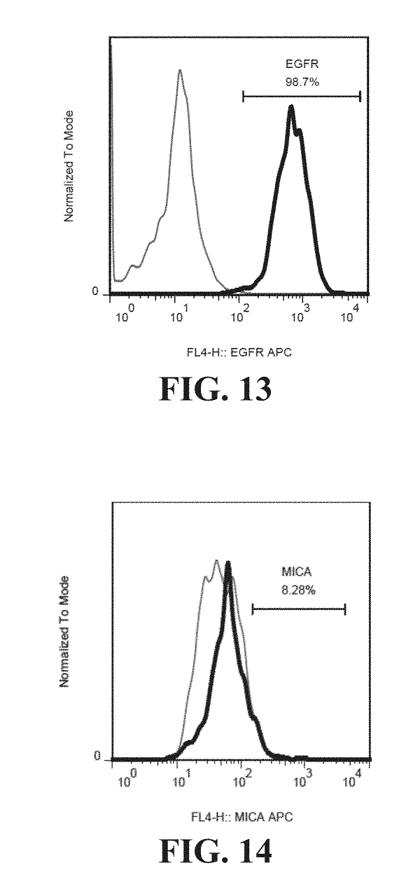

a. Antibody fusion protein obtained from Example 4 can target the tumor cell strains with high-expression EGFR (epidermal growth factor receptor), so the expression of EGFR (epidermal growth factor receptor) on the surface of Hep3B strains (human hepatocellular carcinoma cell, purchased from US ATCC cell bank) is detected by FACS (flow cytometry). The result has shown that the expression rate of EGFR on the surface of human hepatocellular carcinoma cell Hep3B is up to 98.7% (FIG. 13).

b. Antibody fusion protein obtained from Example 4 can express the ligand of NKG2D, and thus increase the ability of NK cells in recognizing and killing tumor cells, so the expression of MICA (the corresponding coding gene sequence is SEQ ID NO:7) on the surface of Hep3B strains is detected by FACS (flow cytometry). The result has shown that the expression rate of MICA on the surface of human hepatocellular carcinoma cell Hep3B is only 8.28% (FIG. 14).

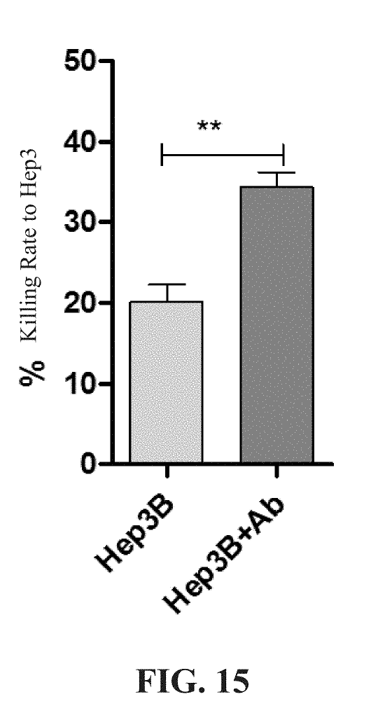

c. Co-culture the NK-92 cells (human NK cell lines, purchased from US ATCC cell bank) and the Hep3B tumor cells in porous cell culture plate, and set control group and antibody fusion protein group (each group is repeated three times; in each hole, the cell number of target cell Hep3B is 1*10⁵ and the cell number of effector cell NK-92 is 5*10⁵). The target cell Hep3B is labeled with TFL-4 and the final concentration is 5 μM, then the cells solution is counted and divided into groups after incubating at 37° C. for 20 min and washing with PBS for 3 times. The control group is prepared by adding effector cell (NK-92 cell), and the antibody fusion protein group is prepared by adding both the effector cell and antibody fusion protein obtained from Example 4 (the adding amount of antibody fusion protein is 12 ng per well), then the mixed solution is incubating at 37° C. for 2 h. After washing the above solution with Washing buffer for 2 times and resuspending the cells with 100 μl buffer, 5 uL annexin FITC is used for staining at room temperature, then 200 μl PBS is added 10 min later, and the positive rate of Annexin V is measured by flow cytometry. Annexin V is a reagent for the detection of apoptosis. Phosphatidyl serine is just distributed in internal area of cell membrane lipid bilayer in normal cells, while the phosphatidyl serine (PS) can move from the inside to the outside in the early stage of apoptosis. As a phospholipids-binding protein, Annexin V has high affinity with phosphatidyl serine, and it can combine with the membrane of early apoptotic cells by phosphatidyl serine exposed outside the cell. So, Annexin V can be seen as a sensitive index for detecting early stage apoptosis. The killing efficiency of NK-92 cells on tumor cells can be indicated by calculating the positive rate of Annexin V. As is shown in the figure, the killing rate of the antibody fusion protein group is almost double that of the control group (FIG. 15).

Finally, it should be stated that it is clear that the above examples are merely used for clearly illustrating the present invention, rather than a limitation of the implementation. For the skilled in the arts, different forms of changes can be made on the basis of the above explanation. There is no need or no way to give exhaustive implements. The obvious changes based on the above are still in the scope of the invention.

1. An antibody fusion protein, characterized in that, comprising an antitumor antigen-specific antibody or a Fab fragment thereof, a single domain antibody or single chain antibody, and further comprising a human NKG2D ligand or ligand fragment;
Said human NKG2D ligand or ligand fragment and the antitumor antigen-specific antibody or Fab fragment thereof and the single domain antibody or single chain antibody are mutually connected by a linker peptide.

2. The antibody fusion protein according to claim 1, characterized in that, the amino acid sequence of said human NKG2D ligand are shown in from SEQ ID NO: 1 to SEQ ID NO: 6.

3. The antibody fusion protein according to claim 1, characterized in that, the coding gene sequence of said human NKG2D ligand are shown in from SEQ ID NO: 7 to SEQ ID NO: 12.

4. The antibody fusion protein according to claim 1, characterized in that, said antitumor antigen-specific antibody is a specific antibody targeting human tumor.

5. A recombinant vector, a recombinant cell, a recombinant bacterium, or an expression cassette, which contains the coding gene of any of the antibody fusion protein according to claim 1.

6. A preparation method of any of the antibody fusion protein according to claim 1, characterized in that, comprising the following steps:
(a) Insert the heavy chain coding gene, NKG2D ligand coding gene and linker peptide coding gene of said antibody fusion protein into pBluescript II SK (+) vector to construct the pBS-SK-H plasmid;
(b) Insert the light chain coding gene of said antibody fusion protein into pBluescript II SK (+) vector to construct the pBS-SK-L plasmid;
(c) Cleave the pBS-SK-H plasmid by enzyme to obtain vector fragment, which contains the heavy chain coding gene, NKG2D ligand coding gene and linker peptide coding gene of said antibody fusion protein;
(d) Cleave the pBS-SK-L plasmid by enzyme to obtain vector fragments, which contains the light chain coding gene of said antibody fusion protein;
(e) Insert the vector fragments obtained in the step (c) and the step (d) into expression vector to obtain the recombinant expression vector;
(f) Transform the recombinant expression vector obtained in the step (e) into recipient cell to express said antibody fusion protein.

7. The preparation method according to claim 6, characterized in that, the expression vector in the step (e) is pcDNA3.0-FLAG.

8. The preparation method according to claim 6, characterized in that, the recipient cell in the step (f) is 293T cell.

9. A use of any of the fusion protein antibody according to claim 1 in preparation of anti-tumor drugs.

10. A drug containing any of the fusion protein antibody according to claim 1.
